(19) United States
(12) Patent Application Publication
(10) Pub. No.: US 2025/0172551 A1
(43) Pub. Date: May 29, 2025

(54) HIGHLY SENSITIVE CARBON NANOTUBE BIOSENSOR WITH REFERENCE ELECTRODE

(71) Applicant: UNIVERSITY OF WASHINGTON, Seattle, WA (US)

(72) Inventors: Jae-Hyun Chung, Seattle, WA (US); Tianyi Li, Seattle, WA (US); Scott Soelberg, Seattle, WA (US); Zachary Taylor, Seattle, WA (US); Clement E. Furlong, Seattle, WA (US); Sang-gyeun Ahn, Seattle, WA (US)

(73) Assignee: UNIVERSITY OF WASHINGTON, Seattle, WA (US)

(21) Appl. No.: 18/840,800

(22) PCT Filed: Feb. 24, 2023
(86) PCT No.: PCT/US2023/063291

§ 371 Date: Aug. 22, 2024

Related U.S. Application Data
(60) Provisional application No. 63/314,244, filed on Feb. 25, 2022.

(51) Int. Cl. G01N 33/543; G01N 33/569

(52) U.S. Cl. CPC G01N 33/5438; G01N 33/56983

(57) ABSTRACT

A system for detecting a biological target in a sample, including a sensor comprised of single-walled nanotubes coating a sample substrate, where the sensor includes a sensing electrode and a control electrode, and an analyzer configured to accept the sensor including a controller configured to measure a resistance of the sensing electrode and a resistance of the control electrode and compare the resistance of the sensing electrode to the resistance of the control electrode. Additionally, a method of detecting a biological target with the system including placing a sample into an analyzer, inserting a sensor made of single-walled carbon nanotubes into the analyzer, wherein the sensor includes a sensing electrode and a control electrode, submerging the sensing electrode into the sample, submerging the sensing electrode into a washing solution, measuring the resistance of the sensing electrode and the control electrode, and comparing the resistances.

### CROSS-REFERENCE TO RELATED APPLICATION

This application claims priority U.S. Provisional Patent No. 63/314244, filed Feb. 25, 2022, the entire contents of which are hereby incorporated by reference for all purposes.

### STATEMENT OF GOVERNMENT LICENSE RIGHTS

This invention was made with government support under Grant No. 1 U01 HL152401-01, awarded by the National Institute of Health. The government has certain rights in the invention.

### BACKGROUND

The emerging SARS-CoV-2 virus, including its reemerging coronavirus variants. continues to pose a serious threat to global public health and to the economy. As of Jan. 3, 2022, the World Health Organization (WHO) reported worldwide 281,808,270 confirmed cases, including a total of 5,411,759 deaths due to SARS-CoV-2 infection caused COVID-19 diseases. The novel coronavirus has impacted industry, economy, and many facets of our daily life. Currently, COVID-19 continues to spread rapidly in communities around the world.

For the rapid screening of COVID 19, various detection methods have been developed to identify SARS-CoV-2 infection for COVID-19 disease management and treatment. Commercially available methods include both rapid antigen tests and nucleic acid detection methods. Quantitative reverse transcription-polymerase chain reaction (qRT-PCR) assay is considered the gold standard for COVID-19 screening. Significant progress has been made in nucleic acid testing to detect various regions of SARS-CoV-2 genomic RNA from swab samples. These methods are currently used for screening patients for COVID-19 infection. However, these commercial assays require 30 minutes to 6 hours of assay time with expensive and bulky equipment available in well-equipped central laboratories with skilled technicians. Rapid antigen tests recognize SARS-CoV-2 viral proteins using lateral flow immunoassays combined with fluorescent or electrochemical methods. This detection scheme is simple, fast, and low cost. However, rapid antigen tests are limited in sensitivity. In particular, the assays are not sensitive enough to detect the target virus in nasal swab samples at an early stage of infection.

Single-walled carbon nanotubes (SWCNTs) are one of the potential candidates for simple and inexpensive detection of the target analyte binding with high sensitivity and specificity. In some embodiments, the target analyte is a microorganism, a virus, a protein, or a nucleic acid. Resistive SWCNT sensors can detect target binding by electrostatic interaction or work function modification. Viral particles and bacteria can be detected by monitoring this resistance change. Using similar technology, the lower limit of detection (LLD) of the swine influenza virus (H1N1) was 177 TCID50 (50% tissue culture infective dose)/mL. SWCNTs functionalized with heparin detected dengue virus at concentrations as low as 840 TCID50/mL. The LLD was 1 plaque-forming unit (PFU)/mL for detecting H1N1. In our previous report, MTB could be detected at 100 CFU/mL in sputum samples using SWCNT sensors combined with magnetic particles. However, these assays did not demonstrate enough sensitivity to screen early-stage SARS-CoV-2 patients. Since the sensor substrate was made on silicon chips, the fabrication and integration costs were too high using this material for an inexpensive screening assay. Accordingly, methods and devices for rapid screening of COVID-19 are needed.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In one aspect, a system for detecting a biological target in a sample is disclosed, including a sensor made of single-walled nanotubes coating a sample substrate, where the sensor includes a sensing electrode and a control electrode: and an analyzer configured to accept the sensor including a controller configured to measure a resistance of the sensing electrode and a resistance of the control electrode and compare the resistance of the sensing electrode to the resistance of the control electrode.

In some embodiments, the system further includes a sample cup configured to hold the sample. In some embodiments, the sample cup includes a first compartment for holding the sample, and a second compartment for holding a washing solution.

In some embodiments, the system further includes a first motor configured to wash the sensing electrode in the washing solution or the sample. In some embodiments, the system further includes a second motor configured to move sensor up and down, so that the sensing electrode is submerged into the sample or the washing solution. In some embodiments, the system further comprises an air pump configured to dry the sample sensor after the sample sensor is dipped into the washing solution. In some embodiments, the washing solution is DI water.

In some embodiments, the system further comprises a heater configured to keep the sample at a constant temperature.

In some embodiments, the sample is a swab solution, such as a nasal swab solution. In some embodiments, the biological target is a virus, a microorganism, a protein, or a nucleic acid. In some embodiments, the virus is a variant of SARS-CoV-2.

In some embodiments, the analyzer further comprises a voltage source configured to apply a voltage to the sensor while the sensor is submerged in the washing solution. In some embodiments, the analyzer is further configured to measure a difference in a transient voltage value of the sensing electrode and the control electrode.

In another aspect, a method of detecting a biological target with the system described herein is disclosed, including placing a sample into an analyzer, inserting a sensor made of single-walled carbon nanotubes into the analyzer, wherein the sensor comprises a sensing electrode and a control electrode, submerging the sensing electrode into the sample, submerging the sensing electrode into a washing solution, measuring the resistance of the sensing electrode and the control electrode, and comparing the resistance of the sensing electrode to the resistance of the control electrode.

In some embodiments, the method further includes drying the sensor after submerging the sensing electrode into the washing solution.

In some embodiments, the method further includes, while the sensing electrode is submerged in the washing solution, applying a voltage to the sensor. In such embodiments, comparing the resistance of the sensing electrode to the resistance of the control electrode further comprises measuring a difference in a transient voltage value of the sensing electrode and the control electrode.

In some embodiments, the method further includes repeating the steps of submerging the sensing electrode into the washing solution and measuring the resistance of both the sensing electrode and the control electrode two or more times. In some embodiments, the method further comprises averaging the two or more sensing electrode resistances to obtain an averaged sensing resistance. In some embodiments, the method further includes averaging the two or more control electrode resistances to obtain an averaged control resistance. In some embodiments, the method further includes comparing the averaged sensing resistance and the averaged control resistance.

In some embodiments, the method further includes using a compared average resistance to generate one or more parameters to improve the clinical sensitivity and specificity of the method.

In some embodiments, the biological target is a microorganism, a virus, a protein, or a nucleic acid.

In some embodiments, the sensing electrode is functionalized with one or more antibodies for a target analyte, and the control electrode is functionalized with one or more antibodies or BSA that do not react to the target analyte. In some embodiments, the target analyte is a microorganism, a virus, a protein, or a nucleic acid.

In some embodiments, the sensing electrode and the control electrode are silk-screened with conductive ink.

In some embodiments, the method further comprises agitating the sample or the washing solution with the sensing electrode to improve the reaction or enhance the washing.

In another aspect, a system for detecting a biological target in a sample is disclosed, including a sensor comprised of single-walled nanotubes coating a sample substrate, wherein the sensor comprises a sensing electrode and a control electrode. In some embodiments, the system includes an analyzer. The analyzer is configured to accept the sensor and includes a voltage source configured to apply a periodic voltage signal to the sensor, while the sensor is submerged in the sample, and a controller configured to measure an impedance of the sensing electrode and an impedance of the control electrode and compare the impedance of the sensing electrode to the impedance of the control electrode.

In yet another aspect, a method of detecting a biological target with the system described herein is disclosed. In some embodiments, the method includes placing the sample into the analyzer, inserting the sensor made of single-walled carbon nanotubes into the analyzer, wherein the sensor comprises the sensing electrode and the control electrode, submerging the sensing electrode into the sample, applying a periodic voltage to the sensor, measuring the impedance of the sensing electrode and the control electrode, and comparing the impedance of the sensing electrode to the impedance of the control electrode.

### DESCRIPTION OF THE DRAWINGS

The foregoing aspects and many of the attendant advantages of this technology will become more readily appreciated as the same become better understood by reference to the following detailed description, when taken in conjunction with the accompanying drawings, wherein:

FIG. 1A is an example device, in accordance with the present technology;

FIG. 1B is an example SWCNT sensor, in accordance with the present technology;

FIGURE 1C is an example SWCNT sensor, in accordance with the present technology;

FIGURE 1D is an example sample cup and solution well, in accordance with the present technology;

FIG. 1E is an example electric circuit for use with the device of FIG. 1A, in accordance with the present technology;

FIG. 2A is an example SWCNT sensor, in accordance with the present technology;

FIG. 2B is an example method of functionalizing antibodies with a device, in accordance with the present technology;

FIG. 2C is a graph of antibody characterizations using ELISA, in accordance with the present technology;

FIGS. 3A-3F are example steps for using the device of FIG. 1A, in accordance with the present technology;

FIGS. 4A-4H are graphs showing resistance changes in the sensors with and without the presence of antibodies, in accordance with the present technology;

FIG. 5A is a graph showing resistance changes for negative samples, in accordance with the present technology;

FIG. 5B is a graph showing resistance changes for positive samples, in accordance with the present technology;

FIGS. 5C-5E are graphs showing combined scores for various concentrations of SARS-CoV-2, in accordance with the present technology;

FIG. 5F is a graph of average resistances at different concentrations of SARS-CoV-2; in accordance with the present technology;

FIG. 6 is a graph of combined scores for negative and positive nasal swabs for multiple conditions; in accordance with the present technology;

FIG. 7 is a graph of the sensitivity of reference sensors in contrast with the sensor in accordance with the present technology;

FIG. 8 is an example kit for use with the device of FIG. 1A, in accordance with the present technology;

FIG. 9A is an example voltage applied to a sample, in accordance with the present technology;

FIG. 9B is an example measured voltage; in accordance with the present technology;

FIG. 10A-10B are graphs demonstrating the difference of voltage signals between a control and a sample, in accordance with the present technology;

FIG. 11A shows a graph of normalized difference in voltage signals of a negative sample, in accordance with the present technology;

FIG. 11B shows a graph of normalized difference in voltage signals of a positive sample, in accordance with the present technology; and

FIG. 12 shows a graph of six samples subjected to periodic voltage, in accordance with the present technology.

### DETAILED DESCRIPTION

Disclosed herein is a resistive SWCNT biosensor on a polyethylene terephthalate (PET) film for low-cost COVID-19 screening. Further disclosed is an analyzer configured to accept the biosensor and determine the presence or absence of a biological target. In some embodiments, silver electrodes are silkscreen printed on SWCNTs for large-scale production. The sensitivity and specificity may be characterized for SARS-CoV-2 in phosphate-buffered saline (PBS) and nasal swabs. The relative resistance change of both control and sensor may be measured upon the binding of the spike protein of SARS-COV-2. The SWCNT sensor also can detect a virus in positive nasal swabs previously screened with qRT-PCR. The presented biosensor may facilitate the development of a POC COVID-19 screening platform that has high sensitivity, low cost, and low power requirements. In some embodiments, the presented system can be used to detect any biological target, including a virus, a microorganism, a protein, or a nucleic acid. In some embodiments, The SWCNT immunosensor is designed to handle a minimally processed nasal swab sample suspended in 1 mL of PBS, yielding a ‘Yes or No’ answer.

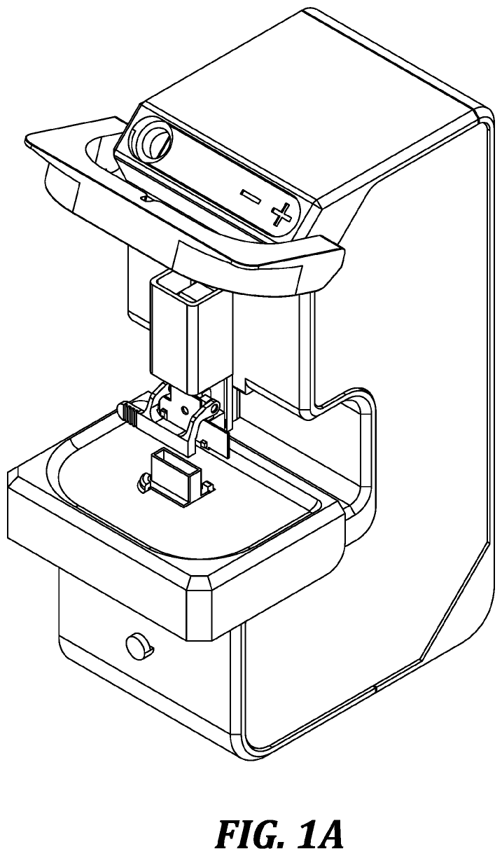

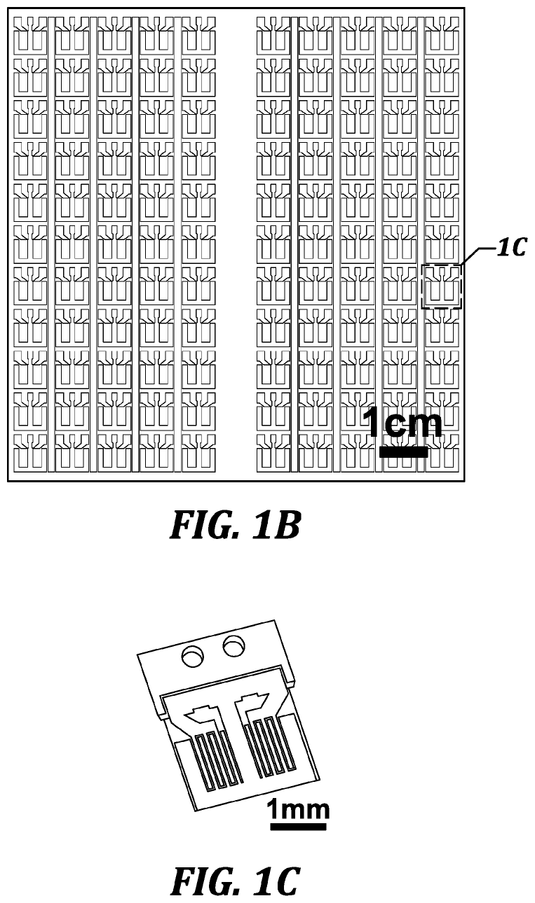

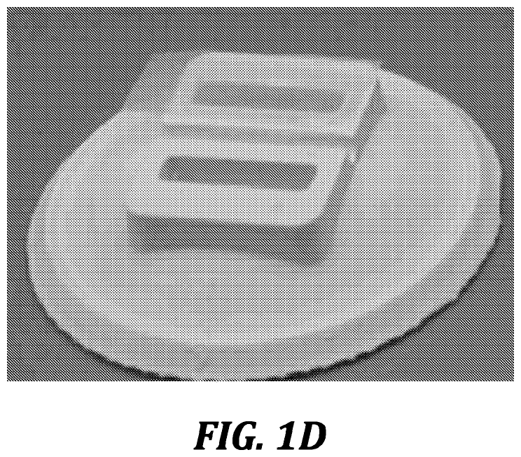

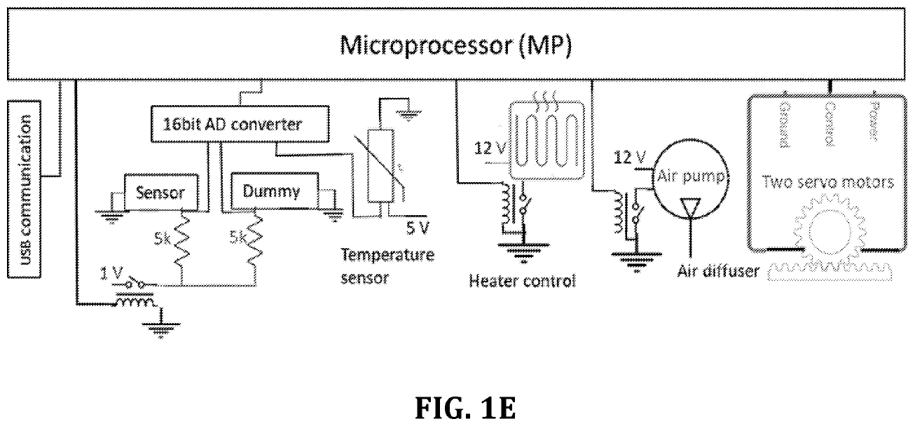

FIG. 1A is an example device, in accordance with the present technology. In one aspect, a system for detecting a biological target in a sample is disclosed, including a sensor made of single-walled nanotubes coating a sample substrate, where the sensor includes a sensing electrode and a control electrode; and an analyzer configured to accept the sensor including a controller configured to measure a resistance of the sensing electrode and a resistance of the control electrode and compare the resistance of the sensing electrode to the resistance of the control electrode.

In some embodiments, the system further includes a sample cup configured to hold the sample. In some embodiments, the sample cup includes a first compartment for holding the sample, and a second compartment for holding a washing solution.

In some embodiments, the system further includes a first motor configured to wash the sensing electrode in the washing solution or the sample. In some embodiments, the system further includes a second motor configured to move sensor up and down, so that the sensing electrode is submerged into the sample or the washing solution. In some embodiments, the system further comprises an air pump configured to dry the sample sensor after the sample sensor is dipped into the washing solution. In some embodiments, the washing solution is DI water.

In some embodiments, the analyzer further comprises a voltage source configured to apply a voltage to the sensor while the sensor is submerged in the washing solution. In some embodiments, the analyzer is further configured to measure a difference in a transient voltage value of the sensing electrode and the control electrode. In some embodiments, the system further comprises a heater configured to keep the sample at a constant temperature.

In some embodiments, the sample is a swab solution. In some embodiments, the sample is a dried blood spot, saliva, or a body solution. As described herein, a body solution is a bodily fluid, such as urine, blood, semen, and the like. In some embodiments, the biological target is a virus, a microorganism, a protein, or a nucleic acid. In some embodiments, the virus is a variant of SARS-CoV-2.

In some embodiments, the device is composed of two linear motors for vertical sensor movement (dipping, withdrawal, and rinsing) and horizontal sample cup movement (solution change and mixing). Resistance may be measured for both sensor and control electrodes. A heater may be embedded under a sample cup to maintain the temperature of about 36° C. to enhance and stabilize the antibody binding and subsequent wash steps.

FIG. 1B is an example SWCNT sensor (or sensor), in accordance with the present technology. An SWCNT sensor may be fabricated by spin-coating SWCNTs onto the PET film and silk-screening silver electrodes over the SWCNT surface.

FIG. 1C is an example SWCNT sensor, in accordance with the present technology. In one example, a sensor was composed of a sensing electrode and a control electrode. The SWCNTs on the sensing electrode were conjugated with monoclonal antibodies specific to the spike protein of SARS-COV-2. The SWCNTs on the control electrode were conjugated with bovine serum albumin (BSA). The interdigitated electrodes offered a large surface area with high sensitivity.

FIG. ID is an example sample cup and solution well, in accordance with the present technology. The sample cup may contain two liquid compartments: one 1×PBS from a nasal swab sample and a washing solution, such as deionized (DI) water for the washing step. For immunocomplex formation, a buffered solution (PBS) may be used. However, since the electrostatic interaction of SWCNTs is one mechanism for detection, the masking effect by ions in the PBS may be reduced by rinsing the sensor with DI water. The vertical motion of the sensor (as shown in FIG. 1A) plays two roles in detection. One is to eliminate nonspecific binding, and the other is to rinse the sensor.

In some embodiments, the vertical motion of the first motor is also configured to dry the sensor completely. By carefully controlling the sensor withdrawal step, the capillary and viscous forces may remove the nonspecifically bound molecules. The sensor surface may be dried by using a low withdrawal speed (such as 1 mm/s). The remaining water drop at the edge of the sensor surface may also be removed by an air diffuser designed to blow air uniformly over the sensor surface. An air pump, having a flow rate, i.e., 4 L/min. may be connected to the air diffuser, which may be powered on at the withdrawal step. The low flow rate of the air pump may be utilized so that aerosols are not generated but the flow rate can still completely dry the sensor surface. A resistance measurement may be used to confirm the complete dryness of the sensor surface.

In some embodiments, this drying step is omitted, and the resistance of the control electrode and the sensing electrode is measured while the sensor is submerged in the washing solution. In such embodiments, a voltage is applied to the sensor, and the resistance measurement is based on a transient voltage value of both the control electrode and the sensing electrode, as described in detail herein.

In some embodiments, the sample cup does not have a solution well. In such embodiments, the sensor is not submerged in a washing solution. In such embodiments, as the sensor is submerged in the sample, a periodic voltage is applied to the sensor, and the impedance of the control electrode and the sensing electrode is measured, as described in detail herein.

FIG. 1E is an example electric circuit (controller) for use with the device of FIG. 1A, in accordance with the present technology. FIG. 1E shows a possible configuration of control and electric units, including a microprocessor, for example, Atmega 328p. The resistance measurement units may be installed to measure the resistance change of the sensor elements. A joule heating element with a temperature sensor may be installed to maintain the temperature of the sample at about 35 to 37 C. In some embodiments, an air pump is controlled with a relay switch. In some embodiments, two servo motors are combined with a rack and pinion gear to provide accurate linear movements (i.e., up to down and left to right).

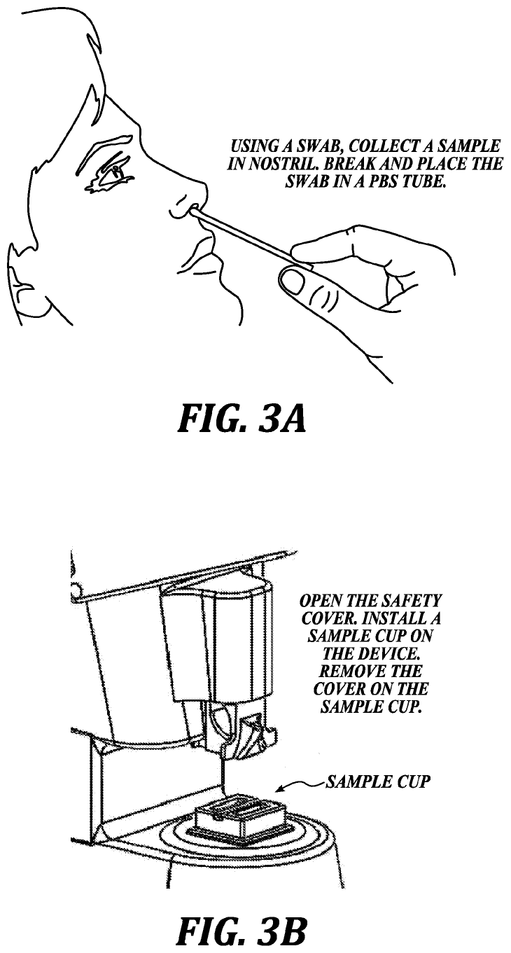

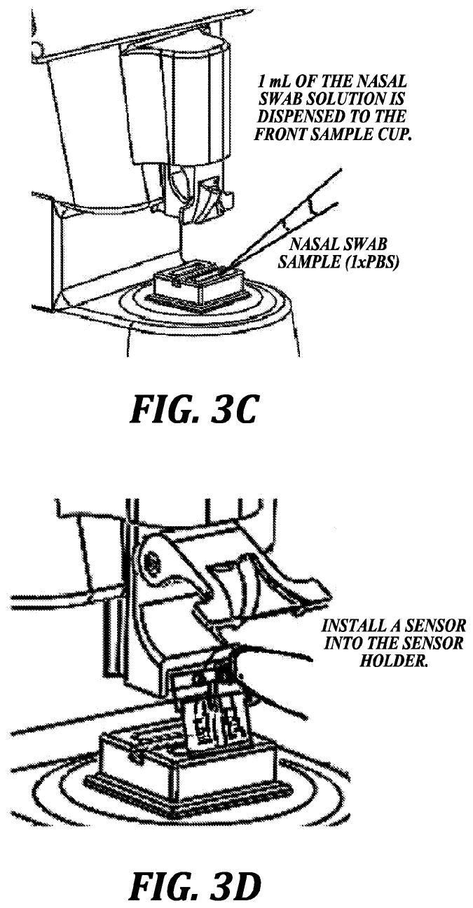

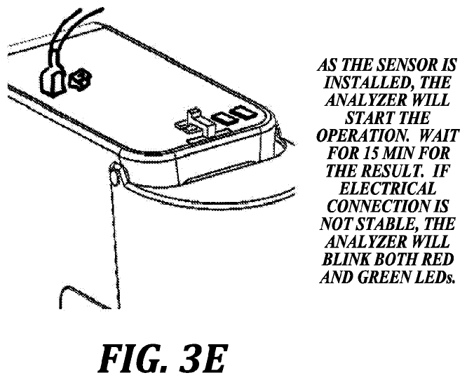

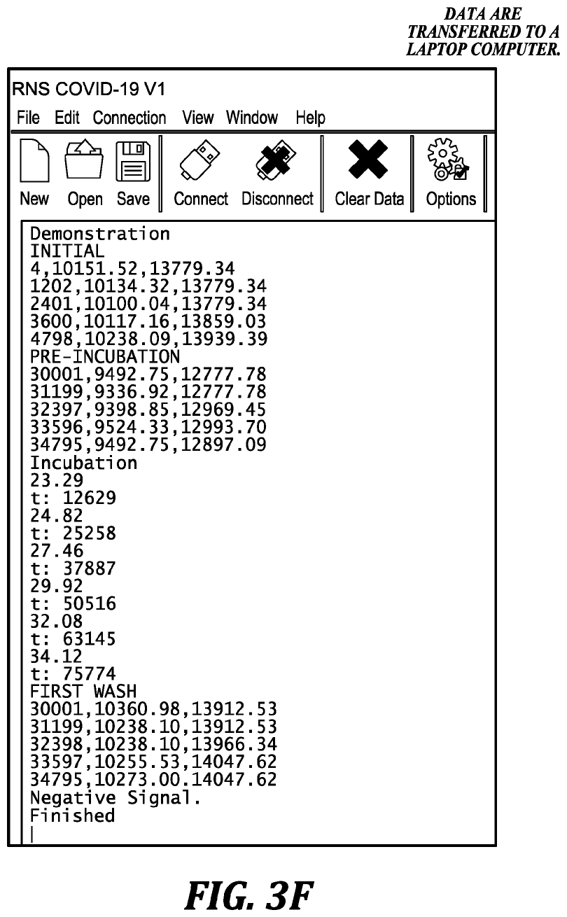

FIGS. 3A-3F are example steps for using the device of FIG. 1A, in accordance with the present technology. For the screening protocol, a swab may be used to collect a sample inside nostrils, as shown in FIG. 3A. The sample swab may then be immersed and stirred in PBS, such as 1.2 mL-1×PBS. A sample cup (such as shown in FIG. 1D) containing a washing solution, such as DI water, may then be installed in the analyzer, as shown in FIG. 3B. In some embodiments, the amount of DI water is 1.1 mL. An amount, such as 1 mL, of the 1×PBS solution containing the swab sample may be transferred to the sample cup, as shown in FIG. 3C. After sensor installation, as shown in FIG. 3D, the analyzer may then initiate a screening protocol. In some embodiments, the screening protocol is a 15 min-screening protocol, as shown in FIG. 3E. In some embodiments, the screening protocol is a 10 min-screening protocol. Once the measurement is completed, the data may be analyzed and transferred to a laptop computer or other communication device, as shown in FIG. 3F.

In another aspect, a method of detecting a biological target with the system described herein is disclosed. The method may include placing a sample into the analyzer as described herein and inserting the sensor made of single-walled carbon nanotubes into the analyzer. As described herein, the sensor may include a sensing electrode and a control electrode. In some embodiments, the method includes submerging the sensing electrode into the sample, submerging the sensing electrode into a washing solution, measuring the resistance of the sensing electrode and the control electrode, and comparing the resistance of the sensing electrode to the resistance of the control electrode.

In some embodiments, the method further comprises drying the sensor after submerging the sensing electrode into the washing solution. In such embodiments, the sensor is dried completely before the resistance measurement of the sensing electrode and the control electrode is taken.

In some embodiments, the method further includes, while the sensing electrode is submerged in the washing solution, applying a voltage to the sensor. In such embodiments, comparing the resistance of the sensing electrode to the resistance of the control electrode comprises measuring a transient value of a measured voltage for the sensing electrode and the control electrode. Further, in such embodiments, the resistance of the control electrode and the sensing electrode is measured while the sensing electrode is submerged in the washing solution.

In some embodiments, the method further includes repeating the steps of submerging the sensing electrode into the washing solution and measuring the resistance of both the sensing electrode and the control electrode two or more times. In such embodiments, the sensor may be dried or submerged in the washing solution while the resistance is measured. In some embodiments, the method further comprises averaging the two or more sensing electrode resistances to obtain an averaged sensing resistance. In some embodiments, the method further includes averaging the two or more control electrode resistances to obtain an averaged control resistance. In some embodiments, the method further includes comparing the averaged sensing resistance and the averaged control resistance.

In some embodiments, the method further includes using a compared average resistance to generate one or more parameters to improve the clinical sensitivity and specificity of the method. In some embodiments, the biological target is a microorganism, a virus, a protein, or a nucleic acid. In some embodiments, the sensing electrode is functionalized with one or more antibodies for a target analyte, and the control electrode is functionalized with one or more antibodies or BSA that do not react to the target analyte. In some embodiments, the target analyte is a microorganism, a virus, a protein, or a nucleic acid.

### Examples

An example system as described herein, and as illustrated in FIG. 1A, was constructed and tested. For the screening protocol, the resistances of the sensing and control electrodes were measured multiple times. In one example the resistances were measured five times during the experimental course of one assay. Example resistance measurement steps are described with the detection protocol in Table 1.

As shown in Table 1, at the initial stage, the resistances of the sensing and control electrodes were measured (R_{0_s} and R_{0_c}). Subsequently, the sensor was immersed into the DI water for 10 seconds, withdrawn, and dried with an air pump for 40 seconds. The resistances were measured after air drying and the five readings were averaged (R_{1_s} and R_{1_c}). The resistance values from DI water showed the initial status of SWCNT sensors. If the ratio (P₁=P_{1_s}/P_{1_c}) was in the range of 0.9˜1.1, the measurement went to the next step. If the ratio was not in the range, the screening was halted due to poor sensor functionality. Once the quality control step was passed, the sensor was then dipped into the sample cup containing the nasal swab sample in 1 mL of 1×PBS. The sensor was agitated in this sample at a speed of 1 mm/second back and forth for 10 minutes with the liquid temperature at 36° C. After a 10 min incubation, the sensor was rinsed in the DI water well at a stirring speed of 2 mm/second for 10 seconds. The sensor was subsequently air-dried, and the resistance values of R_{2_s} and R_{2_c} were measured. The same dipping rinse steps were repeated without stirring twice, during which a set of (R_{3_s}, R_{3_c}) and (R_{4_s}, R_{4_c}) was measured. The three repetitions of the resistance measurement gave stable resistance values.

TABLE 1
Screening protocol, resistance measurement,
parameters, and screening time.

| Resistance | | | Time (min) | |
|---|---|---|---|---|
| Measure- | Resistance Values | | Total = 15 | |
| ment Step | and Ratios | Parameters | min | |
| Before | P₀_{_{—}}₅ = R₀_{_{—}}₅/R₀_{_{—}}₅ and | P₀ = P₀/P₀_{_{—}}_{c} = 1 | 0.5 | min |
| testing | P₀_{_{—}}_{c} = R₀_{_{—}}_{c}/R₀_{_{—}}_{c} | | | |
| Prewash in | P₁_{_{—}}₅ = R₁_{_{—}}₅/R₀_{_{—}}₅ and | P₁ = P₁_{_{—}}_{c} /P₁_{_{—}}_{c} | 1 | min |
| DI water | P₁_{_{—}}_{c} = R₁_{_{—}}_{c}/R₀_{_{—}}_{c} | | | |
| Incubation | N/A | None | 10 | min |
| Wash 1 | P_{2 s} = R_{2 s}/R_{0 s} and | P₂ = P_{2 s}/P_{2 c} | 1 | min |
| | P₂_{_{—}}_{c} = R₂_{_{—}}_{c}/R₀_{_{—}}_{c} | | | |
| Wash 2 | P₃_{_{—}}ₛ = _{R3}_{_{—}}ₛ/R₀_{_{—}}ₛ and | P₃ = P₃_{_{—}}ₛ/P₃_{_{—}}ₛ | 1 | min |
| | P₃_{_{—}}_{c} = R₃_{_{—}}_{c}/R₀_{_{—}}_{c} | | | |
| Wash 3 | P₄_{_{—}}ₛ = R₄_{_{—}}ₛ/R₀_{_{—}}ₛ and | P₄ = P₄_{_{—}}ₛ/P₄_{_{—}}_{c} | 1 | min |

P₄_{_{—}}_{c} = R₄_{_{—}}_{c}/R₀_{_{—}}_{c}

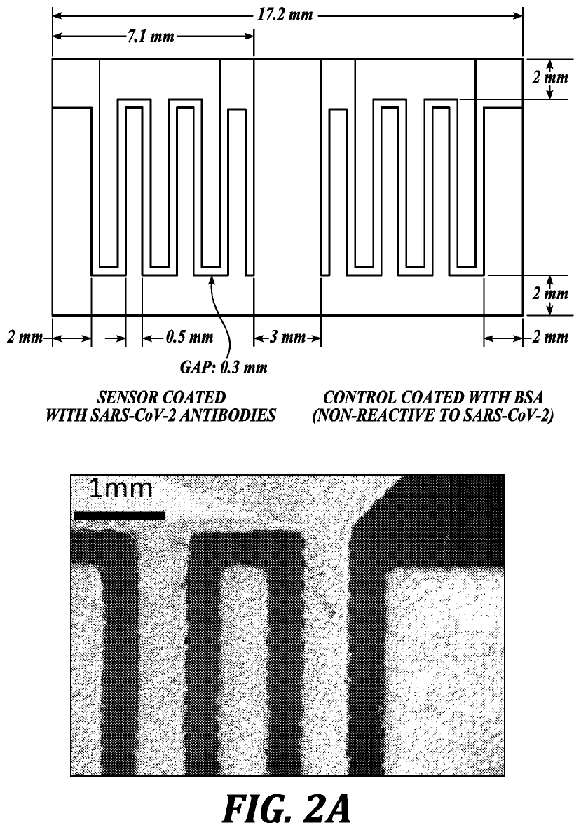

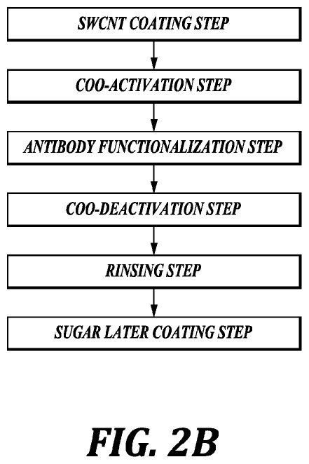

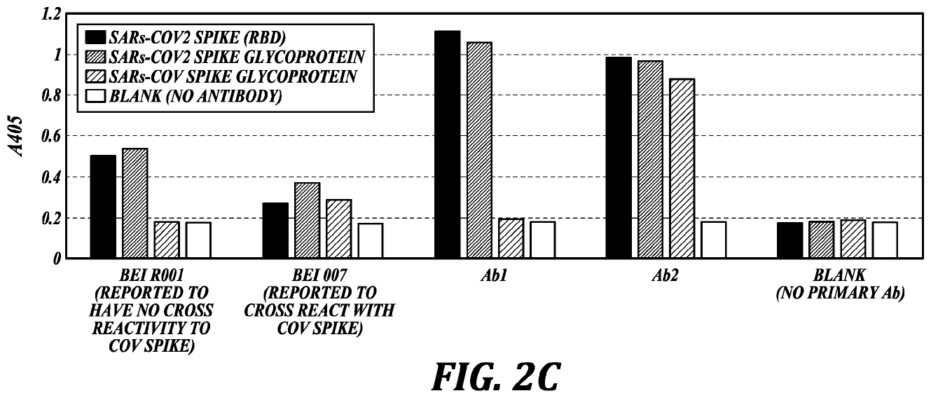

FIG. 2A is an example SWCNT sensor, in accordance with the present technology. In one example, an SWCNT sensor was prepared by screen-printing silver electrodes on an SWCNT-coated polyethylene terephthalate (PET) substrate. Polyethyleneimine solution (0.1% PEI in DI water) was prepared by diluting a stock solution (50%, Millipore-Sigma). The diluted PEI solution was spin-coated on a 100 μm-thick PET film (3M Highland 903) at 3,000 rpm for 3 minutes. The PEI-coated film was cured at 100° C. for 10 minutes. Carboxylic acid-functionalized SWCNTs (SWCNT-COOH, Millipore-Sigma) were dispersed in double-distilled water (ddH2O, Millipore-Sigma) at 0.3 mg/mL. Using a horn-type sonicator, SWCNTs were dispersed for 20 minutes. The SWCNT suspension was spin-coated on a PEI-coated PET film at 3,000 rpm for 3 minutes, followed by curing at 100° C. for 10 minutes. Silver ink was used to silkscreen electrodes on the SWCNT-coated sensor surface. A screen-printing mask was fabricated by Sefar Inc. (NY, USA). The patterned silver ink was cured at 120° C. for 15 minutes. The sensor was composed of two resistive sensing sections; the left section detected the SARS-CoV-2 virus, and the right section served as a control electrode. Both sections contain two interdigitated electrodes whose fingers were separated by 0.3 mm.

FIG. 2B is an example method of functionalizing antibodies with a device, in accordance with the present technology. To covalently immobilize antibodies onto the SWCNT-COOH, a protocol was modified from the reference to activate carboxyls on the SWCNT for covalently bonding to amino groups on antibodies. A solution of 38.5 mg/ml EDC (Thermo #22980) and 11 mg/ml S-NHS (Thermo #PG8-2071) in DI water was prepared. 60 μl of this solution was pipetted onto each side of the sensor and incubated for 15 minutes at room temperature. Sensors were then washed with DI water from a wash bottle and dried with a stream of air from a compressor. In some embodiments, the system is configured to detect a biological target. In some embodiments, the biological target is a virus, a microorganism, a protein, or a nucleic acid. Here, a 20 μg/mL solution of either virus-specific antibody or BSA was added (left side=80 μl Ab, right side=80 μl BSA) and incubated at room temperature for 2 hours. Sensors were then rinsed with DI water and dried with a stream of air from a compressor. To quench any remaining amine-reactive groups, the pH was raised to 8.0 to speed hydrolysis. This was done by adding 300 μl PBS pH 8 to cover both sides of each sensor and incubating overnight at room temperature. Sensors were then rinsed with DI water and dried with a stream of air from a compressor. A layer of trehalose and dextran mixture was added to protect the antibody surface during storage. Each sensor was dipped in a trehalose/dextran solution [2.5% trehalose, 2.5% dextran (average MW 500,000)] to cover the lower ⅔ of the sensing area. Sensors were then cured for 2 H in a 37° C. incubator.

FIG. 2C is a graph of antibody characterizations using ELISA, in accordance with the present technology. Four commercial antibodies were compared for binding to spike protein of both SARS-CoV and SARS-CoV-2. Antibody cross-reactivity was measured to SARS-CoV-2 whole spike protein (BEI NR-52308), SARS-CoV-2 receptor-binding domain (RBD) section of spike protein (Sino Biological 40592-V05H), and SARS coronavirus whole spike (S) Protein (BEI NR-722).

In this example, antibody concentrations were matched with N=2. Two antibodies were previously tested for cross-reactivity with both SARS-CoV and SARS-CoV-2 spike protein (both whole and RBD). Sino Biological 40150-R007 has been previously shown to be specific to the SARS-CoV-2 spike S1 domain and spike receptor-binding domain (RBD) and has also been shown to be cross-reactive with the SARS-CoV Spike S1 domain and RBD. Sino Biological 40150-R001 was specific to the SARS-CoV-2 spike protein RBD as shown previously in ELISA, with cross-reactivity to the SARS-CoV-2 spike S1 protein. However. cross-reactivity was not observed in ELISA with S1 glycoproteins from SARS-CoV.

Protein binding plates (Immulon 2HB, ThermoFisher Scientific 3455) were coated with 100 μL of a 2 μg/mL antigen (spike protein) solution for 24 H at RT. Following antigen binding, plates were washed w/DPBS from a wash bottle and then blocked with a 1 mg/ml BSA solution in DPBS (200 μL) and incubated for 30 min at 37° C. After washing excess BSA from the plate with DPBS from a wash bottle, a solution of the primary antibody (100 μL of 1 μg/mL in DPBS) was added and incubated for 30 min at 37° C. The plate was then washed with DPBS, and a 100 μL of anti-rabbit conjugated HRP (Invitrogen 31460) at a 1:2000 dilution was added and incubated at 37° C. After 30 minutes, the excess secondary antibody was removed by washing with DPBS, then 100 μL ABTS substrate (ThermoFisher 37615) was added and incubated for 10 min at room temperature. The plate was then read for absorbance on a microplate reader at A405 nm.

As shown in FIG. 2C, the ELISA results showed the specific binding of BEI R001 and Ab1 to SARS-CoV-2 whole spike and RBD, while Ab2 shows additional cross-reactivity to SARS-CoV whole spike protein.

The sensor resistance change was characterized by sensors with and without antibodies. According to the observations, the functionalization step to activate and deactivate carboxyl groups on the SWCNT surface dominated the sensor resistance change. When the carboxyl groups were activated, the sensor resistance change was not reliable. By comparing the resistance change of sensors with and without antibodies, deactivation steps could be modified to result in a predictable change of SWCNT resistances. The resistance values for each step were measured as shown in Table 1. Considering the resistance change of the sensor without antibodies as a control, the functionalization protocol for the sensors with antibodies was optimized. The resistance change was compared for each step. For the comparison tests, 1 mL of 1×PBS was used as the target solution with 1.1 mL of DI water. In addition, an initial test was conducted to study the resistance ratio change for 1×PBS, and 1×PBS spiked with SARS-CoV-2 (1,000 particles/mL, equivalent to 1,000 genome copies/mL).

To test the sensitivity. various concentrations of inactivated SARS-CoV-2 [BEI #NR-52287 (Irradiated, Novel Coronavirus, 2019-nCoV/USA-WA1/2020)] in PBS (102˜105 CFU/mL) were suspended in PBS buffer. A 1 mL solution of the prepared virus sample was loaded into a sample cup. After the initial resistance measurement, an SWCNT sensor was dipped in DI water. followed by air-drying and the 2nd resistance measurement. The SWCNT sensor was then immersed in 1 mL of a virus solution for 10 minutes with an agitation (3 mm/s), followed by an air-dry and the first washing. Two more dipping, air drying, and washing steps were repeated to measure all resistance changes. Based on the initial resistance value, all the normalized resistance values were calculated for data processing. For a control experiment, 1×PBS buffer without the target virus was used.

To evaluate the lower limit of detection for SARS-CoV-2, nasal swab samples were collected from deidentified healthy volunteers. After the complete drying of swabs in air, the swab samples were immersed in 1 mL PBS for 1 minute with gentle stirring. Subsequently, 500 μL of the target analyte (SARS-CoV-2) in PBS was mixed with 500 μL of the eluted swab solution. The 1 mL solution was used to test the LLD. The spiked concentrations of SARS-CoV-2 ranged from 10² to 10⁵ particles/mL in steps of 10-fold dilutions. The resistance values were measured and processed as previously described.

An additional sensitivity test was conducted for the SARS-CoV-2 concentrations of 100, 250, 500, and 1,000 particles/mL in order to estimate the limit of detection (LOD). Based on the results, a linear analysis was conducted to estimate the accurate detection limit.

For cross-reactivity study, the response for SARS-CoV-2 (10³ particles/mL) was compared with Staphylococcus epidermidis (S. Epi at 10³ CFU/mL). Mycobacterium tuberculosis (MTB at 10³ CFU/mL), and Staphylococcus aureus at 10³ CFU/mL, respiratory syncytial virus (RSV, 10⁶ particles/mL) and influenza A (H1N1, 10⁶ particles/mL). The nontargeted samples were suspended in 1×PBS, which was mixed with nasal swab samples. Each sample was repeated three times (N=3).

To validate the assay performance, 12 positive and 10 negative patient samples were tested from previously determined RT-qPCR assayed samples. The samples were collected by anterior nares swabs for the Husky Coronavirus Testing research study (IRB) that provided testing to faculty, staff, and students at the University of Washington in Seattle, WA, USA (PMID: 34805425). Dry samples were transported and eluted in 1 ml Tris-EDTA (PMID: 34286830) and stored at −80° C. Among the samples, 12 positive and 10 negative samples were randomly chosen and de-identified for sensor testing. The positive samples included alpha and delta variants of SARS-CoV-2. The sample collection and testing procedure was approved by the institutional review board (IRB) at the University of Washington (Husky Testing number: STUDY00011148).

Since the collected sample volume after RT-qPCR assays was only 100 μL, the sample was diluted to 1 mL using 1×PBS. The 1 mL samples were tested by the prepared SWCNT sensors in a non-blind fashion. RT-qPCR Ct values were determined after thawing and dilution to serve as a direct comparator to the SWCNT.

When the sensor was fabricated without antibodies, the average resistance values were 9.88±1.51 kΩ (N=12). When the antibodies were immobilized on the sensor surface, the average resistance values increased to 24.5±2.69 kΩ. The normalized difference between the sensor and control resistances was 0.037±0.010 for the sensors immobilized without antibodies (N=6). The normalized difference between the sensor and control resistances was −0.057±0.061 for the sensors with immobilized antibodies (N=6). Before antibody immobilization, the normalized resistance difference between sensing and control electrodes was positive. After antibody immobilization, the normalized resistance difference changed to a negative value due to the difference between SARS-CoV-2 antibodies and BSA on the electrodes.

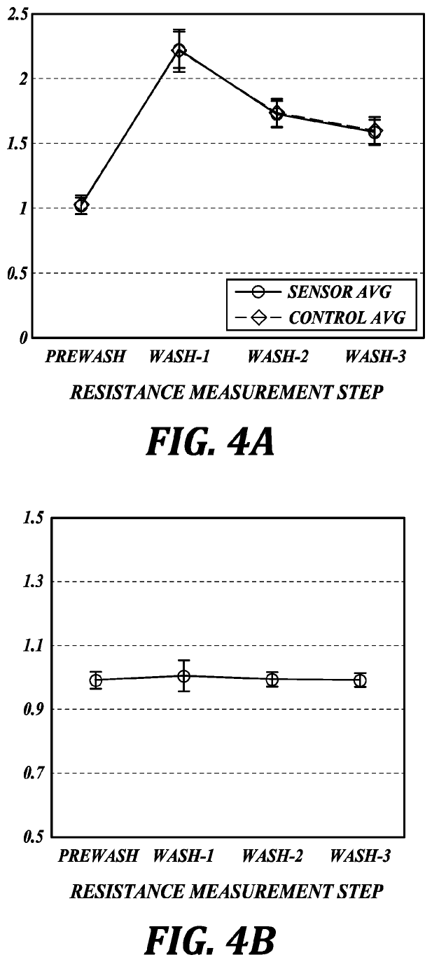

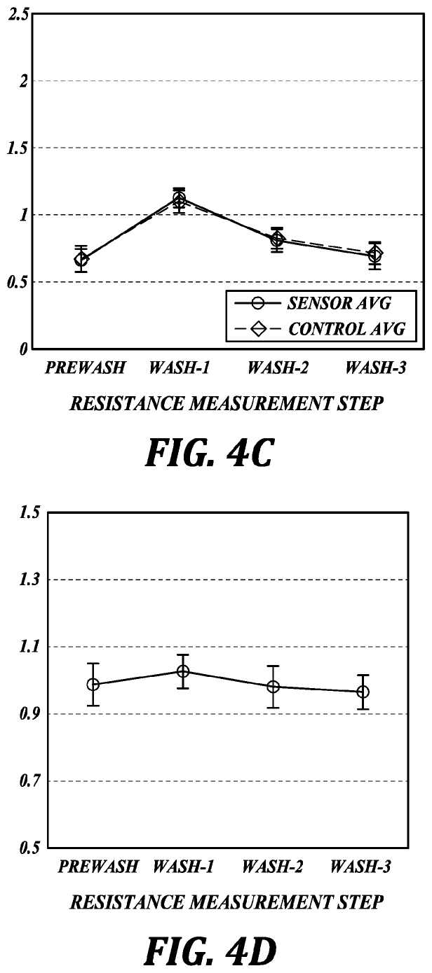

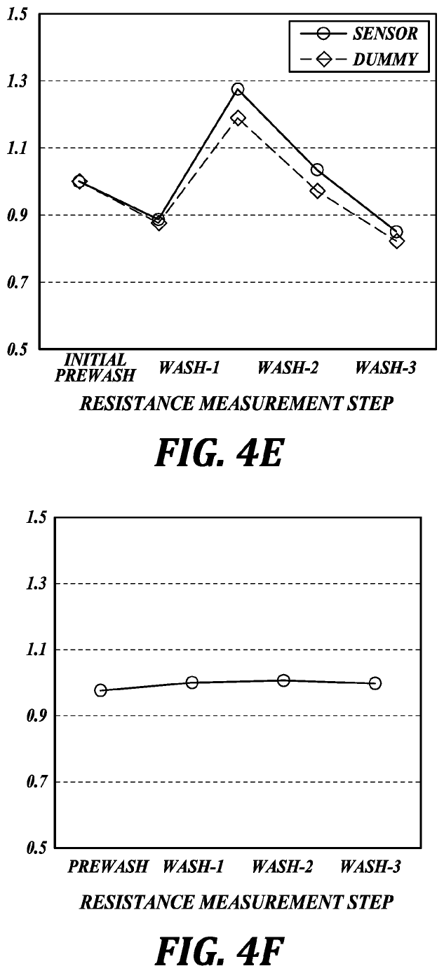

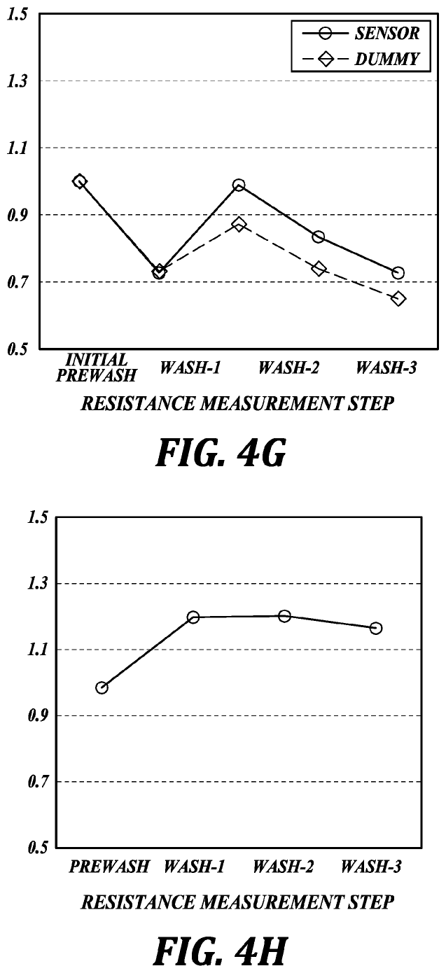

FIGS. 4A-4H are graphs showing resistance changes in the sensors with and without the presence of antibodies, in accordance with the present technology. To study the resistance change for the sensors with and without antibodies, both sensors were tested by the analyzer using the screening protocol. For this experiment, 1×PBS was used without any target analytes. As described in Table 1, the initial resistance values (R₀) were collected from the sensor (R_{0_s}) and control (R_{0_c}) electrodes. The initial resistance values served as a baseline for the following measurements. FIGS. 4A and 4B show P_{i_s} and P_{i_c} value changes, and their ratio change Pᵢ (i=0, 1, 2, 3, and 4) for the sensors without antibodies. The normalized resistances of P_{i_s} and P_{i_c} was close to 1 at the prewash step in DI water, followed by an increase at the first rinsing step. The increase of the normalized resistance values was caused by the ion adsorption on the surface when the sensor was immersed in 1×PBS. The normalized resistance values decreased at the second and third rinsing steps as the ions were depleted in DI water.

FIGS. 4C and 4D present the P_{i_s} and P_{i_c}, and Pᵢ value changes with antibodies, respectively. With immobilization of antibodies, the prewash values of P_{1_s} and P_{1_c} reduced to 0.67, but the overall trend was similar to the sensors without antibodies. The Pᵢ value showing the relative change between the sensing and control electrodes showed an increased response at the first wash. The P₃ and P₄ values corresponding to the second and third rinsing showed a trend converging to 1. According to the initial characterization, the trend for the Pᵢ values with and without antibodies approached 1 with multiple rinsing steps.

Examples of the resistance changes for the negative control in a nasal swab sample are shown in FIGS. 4E and 4F. The P_{i_s} and P_{i_c} values for sensing and control electrodes appeared to diverge more than pure PBS solution (FIG. 4E), but the Pᵢ values between sensor and control remained close to 1 (FIG. 4F). FIGS. 4G and 4H are the example of the resistance changes for the positive control, meaning that 10³ particles/mL in nasal swab samples. When the target viruses were captured on the sensor surface, the P_{i_s} and P_{i_c} became larger at the rinsing steps (FIG. 4G). The Pᵢ values clearly showed the difference between sensing and control electrodes in FIG. 4H. In comparison to the negative control signal in FIG. 4f, the positive control signal in FIG. 4H showed the normalized value >1 due to the resistance change difference for sensing and control electrodes.

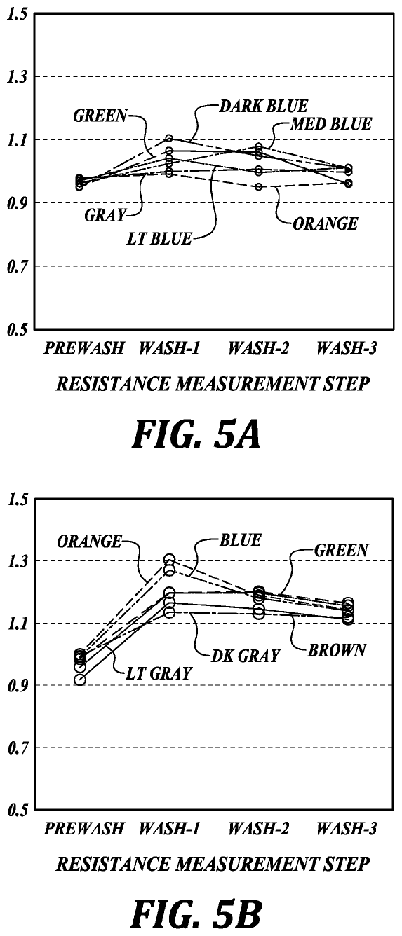

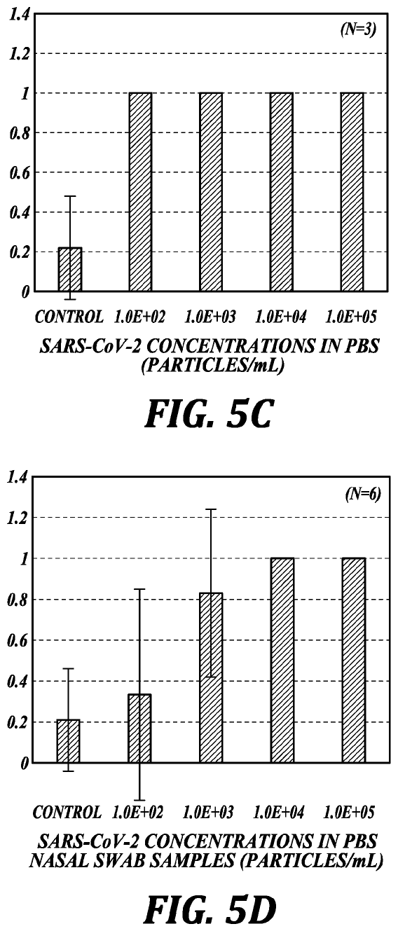

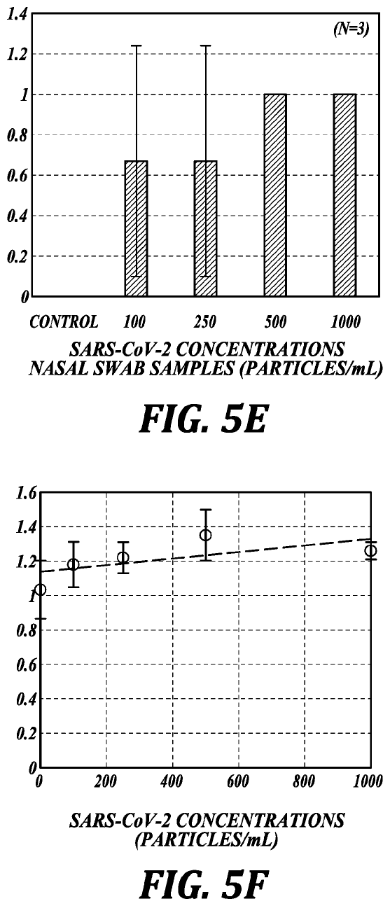

Before the dose response tests in nasal swab samples, the Pᵢ value changes were monitored for negative and positive nasal swab samples to determine the signal processing methods for screening. FIG. 5A is a graph showing resistance changes for negative samples, in accordance with the present technology. FIG. 5B is a graph showing resistance changes for positive samples, in accordance with the present technology. FIG. 5A and 5B show the Pᵢ values for negative swab samples and positive swab samples spiked with 10³ particles/mL-SARS-CoV-2 (N=6), respectively. Overall, the P₂, P₃, and P₄ values of the positive samples were greater than those of the negative samples. However, the Pᵢ values were not clearly differentiated in a certain case, which was attributed to sensor production batches and potential errors in washing steps. It was interestingly found that the positive signals showed a larger slope of Pᵢ between prewash (P₁) and the first wash (P₂). Also, the positive signals showed a lower average slope of (P₂−P₃) and (P₃−P₄). According to the results, we defined two parameters to determine the positive screening results as described in the following conditions:

$$If ( {P}_{2} - {P}_{1} ) > 0.12 , a score of {C}_{1} = 0.5 is given . \qquad ( 1 )$$

$$If ( {P}_{4} - {P}_{1} ) > 0.1 , a score of {C}_{2} = 0.5 is given . \qquad ( 2 )$$

If the combined score of C₁+C₂ was equal to 1, it was positive. If the combined scores were 0 or 0.5, it was negative.

FIGS. 5C-5E are graphs showing combined scores for various concentrations of SARS-CoV-2. in accordance with the present technology. Using the combined scores. the dose response tests of SARS-CoV-2 in PBS were conducted. FIG. 5C shows the combined scores for SARS-CoV-2 in PBS at the concentrations of 10² and 10⁵ particles/mL. All the signals of the positive samples showed the combined value of 1. When the nasal swab samples spiked with 10² and 10⁵ particles/mL were used, two out of six samples showed the combined value of 1 at 10² particles/mL, and five out of six samples showed the combined value of 1 at 10³ particles/mL, as shown in FIG. 5D.

To estimate the lower limit of detection (LLD) in nasal swab samples, further testing was conducted for the SARS-CoV-2 concentrations of 100, 250, 500, and 1,000 particles/mL, as shown in FIG. 5E. At the concentrations of 100 and 250, only one sensor out of three showed the combined score of 1. The combined scores at 500 and 1,000 particles/mL were 1 (N=3). When the average values of P₂, P₃, and P₄ were used, the linear increase of the resistance ratio could be observed, as shown in FIG. 5F). Based on the linear approximation of the average Pᵢ values. the LLD was 350 particles/mL. The LLD of 350 particles/mL was better than typical nucleic amplification-based assays being used for COVID-19 screening. Given that swab samples were replete with human cell fragments, bacteria, and other interferents, these results also demonstrated the specificity of the developed SWCNT sensors.

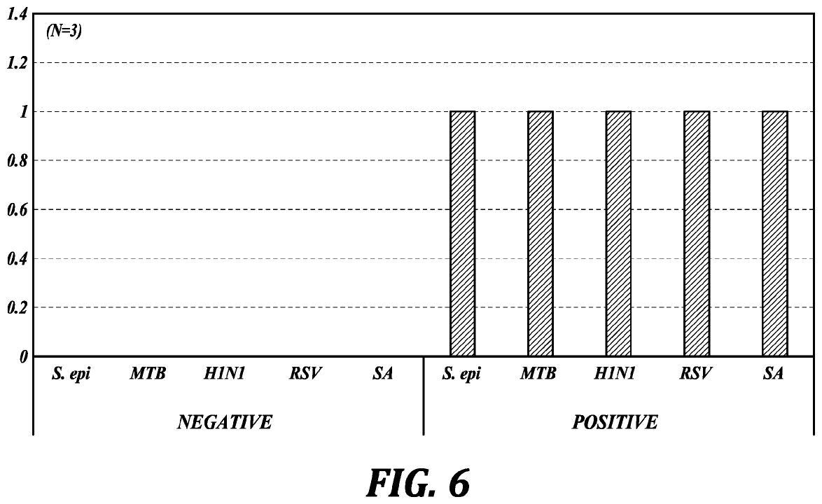

FIG. 6 is a graph of combined scores for negative and positive nasal swabs for multiple conditions, in accordance with the present technology. The average value at 1,000 particles/mL was a little lower than those of other concentrations, which could be caused by the different fabrication batches. Among the dataset, all the sensors but 1,000 particles/mL were from the same batch. Note that the P₂ value was the parameter that was conventionally used for screening using SWCNT sensors. Due to the batch-to-batch variation, the analog value of the resistance ratio could not be used directly. Instead, the combined scores were better to determine the positive and negative screening results.

FIG. 6 shows the cross-reactivity of the SWCNTs sensors. The nasal swab samples spiked with S. epidermidis, MTB, H1N1, RSV, and SA, were used for negative samples. The positive samples were nasal swab samples spiked with 10³ particles/mL-SARS-CoV-2, S. epidermidis, MTB, H1N1, RSV, and SA. The combined scores clearly showed the difference between negative and positive samples.

Clinical testing was performed on previously frozen SARS-CoV2 positive and negative samples collected in TE buffer and tested with RT-PCR. Since only 100 μL of the sample volumes were available in TE buffer, the positive and negative samples were diluted 10-fold using 1×PBS. The PCR test results showed that the positive samples included alpha and delta variants. Among the twelve positive and ten negative samples, an SWCNT sensor showed one false positive result for a negative sample. According to the results, the clinical sensitivity was 100%, and the clinical specificity was 90%.

TABLE 2
Comparison of PCR results and SWCNT sensor results for positive
and negative clinical samples (N/A means Ct > 40).

| | | | | | Reference | |
|---|---|---|---|---|---|---|
| | Reference | Reference | | | PCR | |
| Positive | PCR | PCR | *SWCNT | Negative | Ct Value | *SWCNT |
| Sample ID | Ct Value | Variants | Sensor | Sample ID | (>33) | Sensor |
| XXXX61b | 22.5 | Alpha | Positive | XXXX8ba7 | N/A | Negative |
| XXXXa4dc | 33 | Alpha | Positive | XXXX4fbd | N/A | Negative |
| XXXXf041 | 23.5 | Alpha | Positive | XXXX6ea9 | N/A | Negative |
| XXXX89d7 | 30 | Alpha | Positive | XXXXa467 | N/A | Negative |
| XXXX996 | 29.4 | Alpha | Positive | XXXXd9ee | N/A | Negative |
| XXXXa320 | 38.5 | Alpha | Positive | XXXX297e | N/A | Negative |
| XXXX1b27 | 31.3 | Delta | Positive | XXXX4907 | N/A | **Positive |
| XXXXf10e | 30.6 | Delta | Positive | XXXXde69 | N/A | Negative |
| XXXX8c9c | 26.5 | Delta | Positive | XXXX07e3 | N/A | Negative |
| XXXXf06c | 31.2 | Alpha | Positive | XXXX4d9f | N/A | Negative |
| XXXX5456 | 30.4 | Delta | Positive | | | |
| XXXX76fb | 23.5 | Alpha | Positive | | | |

*Unblinded trial
**A PCR negative sample shows the strong positive signal of the SWCNT sensor
***The sample IDs are deidentified with XXXX

In summary, an immuno-resistive SWCNT sensor was developed to specifically detect SARS-CoV-2 in nasal swab samples. The analytical lower limit of detection was 350 viral particles/mL with the detection time of 15 minutes. The analytical LLD was better than point-of-care screening nucleic acid detection assays. In comparison to other antigen detection assays, the detection limit was 2˜3 orders of magnitude more sensitive. To achieve such high sensitivity and specificity, the relative resistance change of an SWCNT sensor was measured in comparison to a control sensor. To improve the clinical sensitivity and specificity, a combined score using two parameters based on the resistance ratio was used. According to clinical sample tests, the assay showed 100% sensitivity and 90% specificity. The SWCNT sensors detected both alpha and delta variants. The simple resistive measurement will allow rapid screening by minimally trained personnel. Also, a minimal power requirement (<1 W) combined with low assay cost will be important for point-of-care (POC) screening in limited-resource settings.

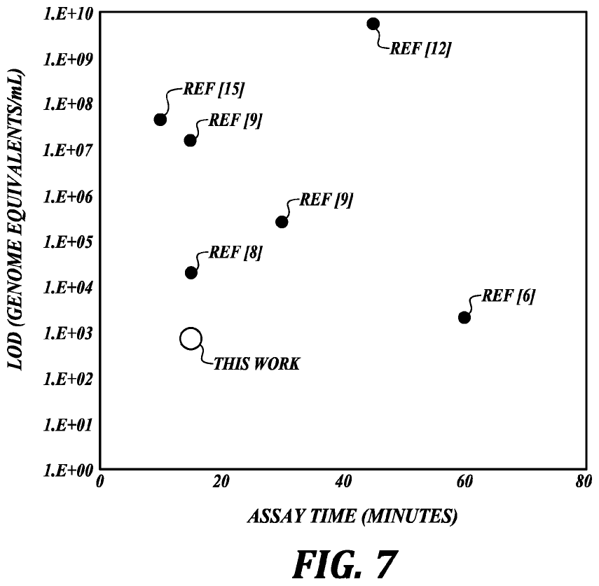

FIG. 7 is a graph of the sensitivity of reference sensors in contrast with the sensor in accordance with the present technology. An example device was compared with comparative devices and methods for rapid COVID-19 testing. On the horizontal axis is the assay time in minutes. On the vertical axis is the LOD in genome equivalents per milliliter. As shown in FIG. 7, the disclosed device was able to detect samples with smaller LOD in less time than the comparative devices.

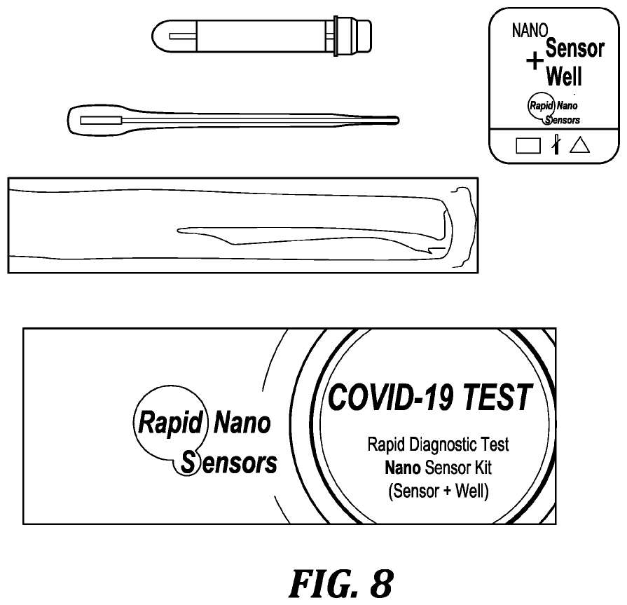

FIG. 8 is an example kit for use with the device of FIG. 1A, in accordance with the present technology. In some embodiments, disclosed herein is a kit for rapid COVID-19 testing, in accordance with the present technology. In some embodiments, the kit includes a packaging containing a testing swab, a solution tube, a dropper, and the sensor as described herein.

In some operation, a user may take a swab of their mouth or nose with the testing swab, add the testing swab into the solution tube, take out a portion or all of the solution in the solution tube with the dropper, and add the solution to the sensor well of the analyzer described herein. The user can then place the sensor well into the analyzer as described herein.

In some embodiments, the washing step may be omitted. In such embodiments, voltage is applied to the sensor as it is submerged in the sample. In some embodiments, system for detecting a biological target in a sample is disclosed, including a sensor comprised of single-walled nanotubes coating a sample substrate, wherein the sensor comprises a sensing electrode and a control electrode. In some embodiments, the system includes an analyzer. The analyzer is configured to accept the sensor and includes a voltage source configured to apply a periodic voltage signal to the sensor, while the sensor is submerged in the sample, and a controller configured to measure an impedance of the sensing electrode and an impedance of the control electrode and compare the impedance of the sensing electrode to the impedance of the control electrode. In some embodiments, a method of detecting a biological target with the system described herein is disclosed. In some embodiments, the method includes placing the sample into the analyzer, inserting the sensor made of single-walled carbon nanotubes into the analyzer, wherein the sensor comprises the sensing electrode and the control electrode, submerging the sensing electrode into the sample, applying a periodic voltage to the sensor, measuring the impedance of the sensing electrode and the control electrode, and comparing the impedance of the sensing electrode to the impedance of the control electrode.

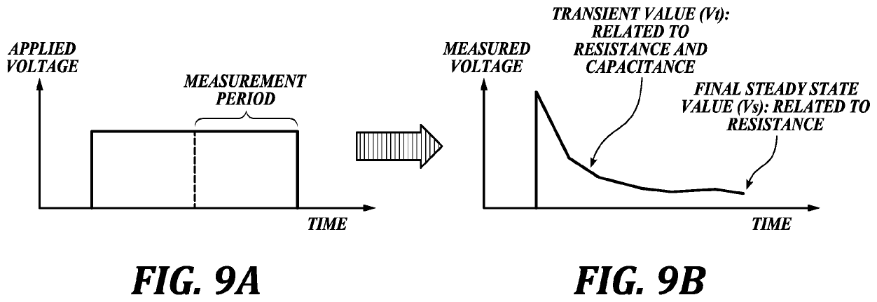

FIG. 9A is an example voltage applied to a sample, in accordance with the present technology. In some embodiments, the device is configured to measure the capacitance of the sensor while the sensor is in the washing solution. In such embodiments, while the sensing electrode is submerged in the washing solution, a voltage is applied to the sensing electrode. In some embodiments, the resistance of the sensing electrode is compared to the resistance of the control electrode by measuring a transient value of a measured voltage. FIG. 9A shows a representative voltage applied to the sample. On the horizontal axis is time, and on the vertical axis is applied voltage. As is shown in FIG. 9A, the voltage applied is a rectangular voltage, but any wave shape may be applied in practice. The voltage shown in FIG. 9A represents a single voltage pulse. In some embodiments, multiple voltage pulses are applied to the sensing electrode over time, and multiple measurements are taken. In some embodiments, about 10 to 20 voltage pulses may be applied.

FIG. 9B is an example measured voltage, in accordance with the present technology. In some embodiments, the device is configured to measure the voltage of each voltage pulse as shown in FIG. 9A. The device may then compare the resistance of the sensing electrode to the resistance of the control electrode by measuring the transient value (Vt) of the measured voltage.

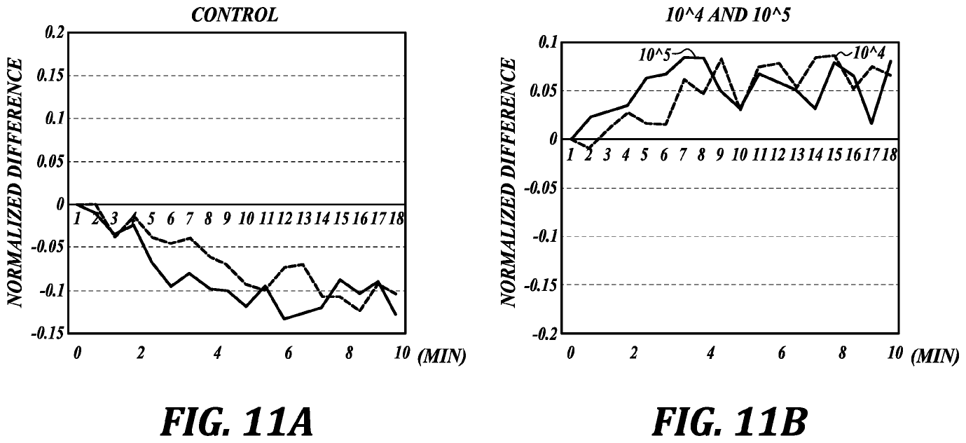

In operation, voltage is applied to the sensor while the sensing electrode is submerged in the washing solution, and a portion of the voltage (i.e., measurement period) is measured. The transient voltage value (Vt) is measured, as opposed to the final steady state value (Vs). The transient voltage value is then factored into the resistance measurement of the control electrode and the sensing electrode. In this manner, the sampling period may be shortened to 10 minutes, as shown in FIGS. 11A and 11B. Further, the resistance may be measured while the sensing electrode is submerged in the washing solution, as opposed to dried. In some embodiments, multiple pulses are applied to the sensing electrode. In some embodiments, 10 to 20 voltage pulses are applied. In such embodiments, the transient voltage measurement can be averaged.

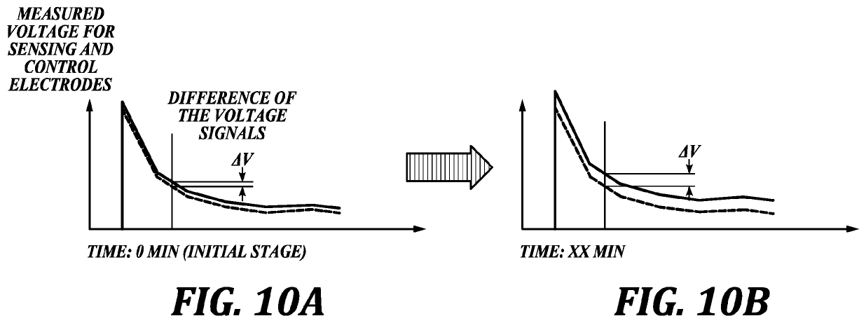

FIG. 10A-10B are two graphs demonstrating the difference of voltage signals between a control (unbroken line) and a sample (dashed line), in accordance with the present technology. On the horizontal axes is the time and on the vertical axes is the measured voltage for the sensing electrode and the control electrode. As shown, in FIG. 10A, when the voltage is first applied, the difference between the voltage signals for the sensing electrode and the control electrode is small, but as time progresses, as shown in FIG. 10B, the difference between the voltage signals becomes larger. In some embodiments, the device described herein is configured to measure the difference in these voltage signals (or Vt) to determine if a sample is positive or negative.

FIG. 11A shows a graph of normalized difference in voltage signals (or transient voltage values) of a negative sample, in accordance with the present technology. On the horizontal axis is the time in minutes. On the vertical axis is the normalized difference between the measured transient voltage values of the control electrode and the sensing electrode. On the horizontal line at zero is the number of times the voltage was measured, for a total of 18 times in 10 minutes. Two negative tests are graphed (unbroken and dashed lines). As shown, the difference between the voltage signals of the control electrode and the sensing electrode trended downwards in both tests, indicating there was a smaller difference between each measured voltage over time. Accordingly, the samples measured with the device were both measured as negative for the presence of the biological target.

In some embodiments, the method further includes, while the sensing electrode is submerged in the sample or washing solution, applying a periodic voltage signal to the sensor, and comparing the impedance of the sensing electrode to the impedance of the control electrode by measuring a difference in a transient voltage value of the sensing electrode and the control electrode while a proton in the sample or washing solution is adsorbed into the single walled carbon nanotubes. In operation, a proton, such as the proton in hydrogen, adheres to the surface of the carbon nanotube surface. The works as an electrostatic gating effect, which increases the sensitivity of the sensor.

FIG. 11B shows a graph of normalized difference in voltage signals (or transient voltage values) of a positive sample, in accordance with the present technology. On the horizontal axis is the time in minutes. On the vertical axis is the normalized difference between the measured voltage signals of the control electrode and the sensing electrode. On the horizontal line at zero is the number of times the voltage was measured, for a total of 18 times in 10 minutes. Two samples were tested, a sample having 10⁴ particles per milliliter of biological target (dashed line), and a sample having 10⁵ particles per milliliter of biological target. As shown, the difference between the voltage signals of the control electrode and the sensing electrode trended upwards in both tests, indicating there was a larger difference between each measured voltage over time. Accordingly, the samples measured with the device were both measured as positive for the presence of the biological target.

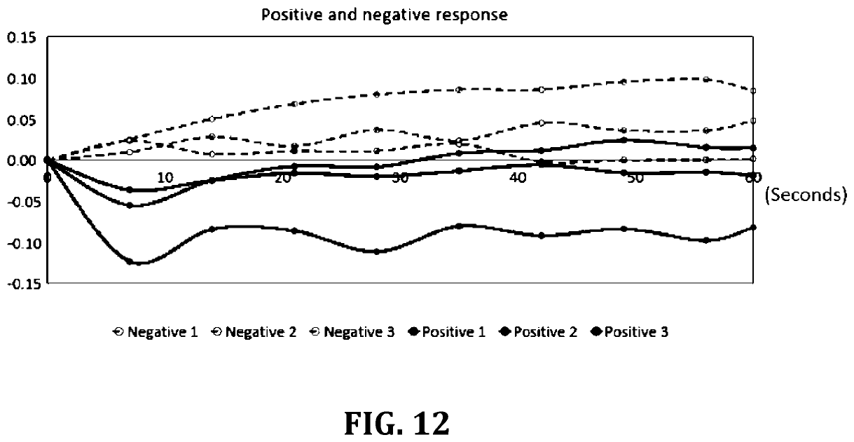

FIG. 12 shows a graph of six samples subjected to periodic voltage, in accordance with the present technology. Three negative samples (dashed lines) and three positive samples (solid lines) are graphed. Each positive sample had a viral concentration of 10\hat{ }5 particles/milliliter. On the horizontal axis is the time in seconds. On the vertical axis is the normalized difference in impedances of the control electrode and the sensing electrode.

In some embodiments, the sensor is submerged in the sample as the periodic voltage is applied. Every ten seconds, the system applied a voltage to the sensor, and measured the impedance of the control electrode and the sensing electrode. In such embodiments, the sensor is not washed in the washing solution. In some embodiments, voltage is applied for five seconds every ten seconds. The amount of time the impedance is measured is referred to as the sampling period (or screening time). As shown in FIG. 12, over one minute (60 seconds) the normalized difference was lower (i.e., >0.05) for positive samples and higher (i.e., <0.0) for negative samples. In some embodiments, the sampling period is one minute. In some embodiments, the sampling period may be three minutes. In some embodiments, the sampling time is 30 seconds. As hydrogen is desorbed from the sensor surface, the sensitivity of the sensor increases.

The use of the word “a” or “an” when used in conjunction with the term “comprising” may mean “one”, but it is also consistent with the meaning of “one or more”, “at least one”, and “one or more than one”. The use of the term “or” is used to mean “and/or” unless explicitly indicated to refer to alternatives only if the alternatives are mutually exclusive, although the disclosure supports a definition that refers to only alternatives “and/or”. Throughout this application, the term “about” is used to indicate that a value includes the inherent variation of error for the quantifying device, the method being employed to determine the value, or the variation that exists among the study subjects. For example, but not by way of limitation, when the term “about” is utilized, the designation value may vary by plus or minus twelve percent, or eleven percent, or ten percent, or nine percent, or eight percent, or seven percent, or six percent, or five percent, or four percent, or three percent, or two percent, or one percent. The use of the term “at least one” will be understood to include one as well as any quantity more than one, including but not limited to, 2, 3, 4, 5, 10, 15, 20, 30, 40, 50, 100, etc. The term “at least one” may extend up to 100 or 1000 or more, depending on the term to which it is attached; in addition, the quantities of 100/1000 are not to be considered limiting, as lower or higher limits may also produce satisfactory results. In addition, the use of the term “at least one of X, Y, and Z” will be understood to include X alone, Y alone, and Z alone, as well as any combination of X, Y, and Z. The use of ordinal number terminology (i.e., “first”, “second”, “third”, “fourth”, etc.) is solely for the purpose of differentiating between two or more items and is not meant to imply any sequence or order or importance to one item over another or any order of addition, for example.

While illustrative embodiments have been illustrated and described, it will be appreciated that various changes can be made therein without departing from the spirit and scope of the technology.

1. A system for detecting a biological target in a sample, comprising:
a sensor comprised of single-walled nanotubes coating a sample substrate, wherein the sensor comprises a sensing electrode and a control electrode; and
an analyzer configured to accept the sensor comprising:
a controller configured to measure a resistance of the sensing electrode and a resistance of the control electrode and compare the resistance of the sensing electrode to the resistance of the control electrode.

2. The system of claim 1, wherein the system further comprises a sample cup configured to hold the sample, wherein the sample cup comprises a first compartment for holding the sample, and a second compartment for holding a washing solution.

3. (canceled)

4. The system of claim 2, wherein the system further comprises:
a first motor configured to wash the sensing electrode in the washing solution or the sample; and
a second motor configured to move sensor up and down, so that the sensing electrode is submerged into the sample or the washing solution.

5. (canceled)

6. The system of claim 4, wherein the analyzer further comprises an air pump configured to dry the sample sensor after the sample sensor is dipped into the washing solution.

7. The system of claim 1, wherein the analyzer further comprises a heater configured to keep the sample at a constant temperature.

8-11. (canceled)

12. The system of claim 3, wherein the analyzer further comprises a voltage source configured to apply a periodic voltage signal to the sensor while the sensor is submerged in the sample or washing solution.

13. The system of claim 12, wherein the analyzer is further configured to measure a difference in a transient voltage value of the sensing electrode and the control electrode.

14. A method of detecting a biological target with the system of claim 1, the method comprising:
placing the sample into the analyzer;
inserting the sensor made of single-walled carbon nanotubes into the analyzer, wherein the sensor comprises the sensing electrode and the control electrode;
submerging the sensing electrode into the sample;
measuring the resistance of the sensing electrode and the control electrode; and
comparing the resistance of the sensing electrode to the resistance of the control electrode.

15. The method of claim 14, wherein the method further comprises submerging the sensing electrode into a washing solution.

16. The method of claim 15, wherein the method further comprises drying the sensor after submerging the sensing electrode into the washing solution.

17. The method of claim 15, wherein the method further comprises:
while the sensing electrode is submerged in the sample or washing solution, applying a voltage to the sensor; and
comparing an impedance of the sensing electrode to an impedance of the control electrode by measuring a difference in a transient voltage value of the sensing electrode and the control electrode.

18. The method of claim 15, wherein the method further comprises:
while the sensing electrode is submerged in the sample or washing solution;
applying a periodic voltage signal to the sensor; and
comparing an impedance of the sensing electrode to an impedance of the control electrode by measuring a difference in a transient voltage value of the sensing electrode and the control electrode while a proton in the sample or washing solution is adsorbed into the single walled carbon nanotubes.

19. The method of claim 15, wherein the method further comprises:
repeating the steps of submerging the sensing electrode into the washing solution and measuring the resistance of both the sensing electrode and the control electrode two or more times.

20. The method of claim 19, wherein the method further comprises averaging the two or more sensing electrode resistances to obtain an averaged sensing resistance.

21. The method of claim 19, wherein the method further comprises averaging the two or more control electrode resistances to obtain an averaged control resistance.

22. The method of claim 19, wherein the method further comprises comparing the averaged sensing resistance and the averaged control resistance.

23-24. (canceled)

25. The method of claim 14, wherein the sensing electrode is functionalized with one or more antibodies for a target analyte, and the control electrode is functionalized with one or more antibodies or BSA that do not react to the target analyte.

26-27. (canceled)

28. The method of claim 14, wherein the method further comprises agitating the sample or the washing solution with the sensing electrode to improve the reaction or enhance the washing.

29. (canceled)

30. A method of detecting a biological target with the system of any claim 1, the method comprising:
placing the sample into the analyzer;
inserting the sensor made of single-walled carbon nanotubes into the analyzer, wherein the sensor comprises the sensing electrode and the control electrode;
submerging the sensing electrode into the sample;
applying a periodic voltage to the sensor;
measuring the impedance of the sensing electrode and the control electrode; and
comparing the impedance of the sensing electrode to the impedance of the control electrode.

31. The method of claim 30, wherein the periodic voltage is applied for about 30 seconds to about 3 minutes.

32-33. (canceled)
